(19) United States
(12) Patent Application Publication
(10) Pub. No.: US 2015/0359972 A1
(43) Pub. Date: Dec. 17, 2015

(54) SYRINGE CAP REMOVING DEVICE

(71) Applicant: YUE PFONG INTERNATIONAL TECHNOLOGY CORP., Taichung (TW)

(72) Inventor: Te-Peng HUANG, Taichung (TW)

(21) Appl. No.: 14/302,367

(22) Filed: Jun. 11, 2014

(51) Int. Cl. A61M 5/32

(52) U.S. Cl. CPC A61M 5/321

(57) ABSTRACT

A syringe cap removing device of the present invention includes a seat, a main body, and a clamp. The main body is detachably disposed on the seat with an end and. The clamp is disposed on an end of the main body opposite to the seat. The clamp defines a clamping space for clamping a syringe clamp. Thus, the clamp can be positioned on the seat or be detached from the seat for held by hand so that the present invention helps user remove the syringe cap without touching it directly.

### BACKGROUND OF THE INVENTION

1. Field of the Invention

The present invention is related to a syringe cap removing device, more especially to a removing device which clamps the syringe cap.

2. Description of the Prior Art

Medical workers touch and use plural syringes everyday, so they are in danger of infection by needle pricking. Thus, a used syringe needle should be covered by a cap and be removed form the syringe. However, the needle is extremely thin, and the opening of the cap is also tiny. As a result, it is difficult to insert the needle into the cap.

To prevent needle pricking, a clamping device can position the cap, and the user can hold the syringe to insert the needle into the cap without holding the cap by hand. As disclosed in patents TW 312155 and TW 363423, the clamping device is held with one hand, and the syringe is held with the other hand. That is, the device is unable to be use with a single hand. Patent TW 479524 disclosed a device which is able to be positioned on a table or others, so the device is possibly used with a single hand. However, the direction of the clamping portion can be adjusted limitedly. In addition, to release the cap into a collecting container, the seat with the clamping device has to be removed from the table.

### SUMMARY OF THE INVENTION

The main object of the present invention is to provide syringe cap removing device having a clamp which is able to be positioned on a seat or to be held by hand directly.

To achieve the above and other objects, syringe cap removing device, including a seat, a main body, and a clamp.

An end of the main body is detachably disposed on the seat. An end of the clamp is disposed on an end of the main body opposite to the seat. The clamp defines a clamping space for clamping a syringe cap.

Thereby, the removing device of the present invention can be positioned on a table or a rack, or be detached for held by hand. Thus, the clamp can grasp the syringe cap so that a user can remove the syringe needle from the syringe to keep the needle in the cap. And then, the clamp is released at an appropriate place for collecting the cap and the needle. As a result, the cap and the needle may not be touched by the user so as to prevent infection.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of the present invention;

FIG. 2 is a breakdown drawing of the present invention;

FIG. 3 is a top view of the present invention;

FIG. 4 is a top view of the present invention when a clamp is opened;

FIGS. 5 to 7 are illustrations of the present invention;

FIG. 8 is a stereogram showing a second embodiment of the present invention;

FIG. 9 is an illustration of a second embodiment of the present invention.

### DETAILED DESCRIPTION OF THE PREFERRED EMBODIMENTS

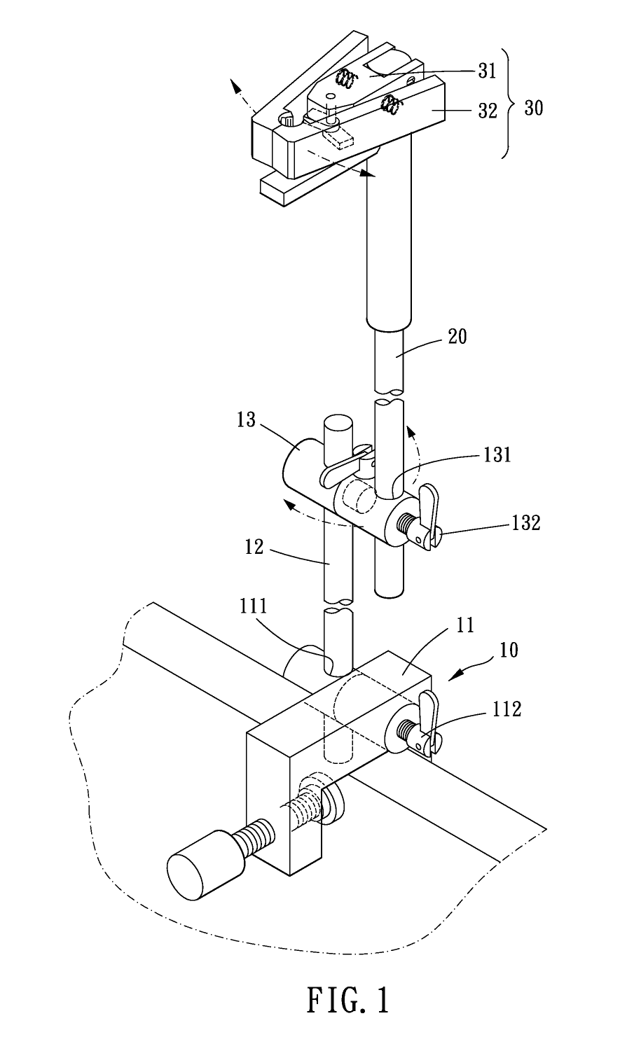

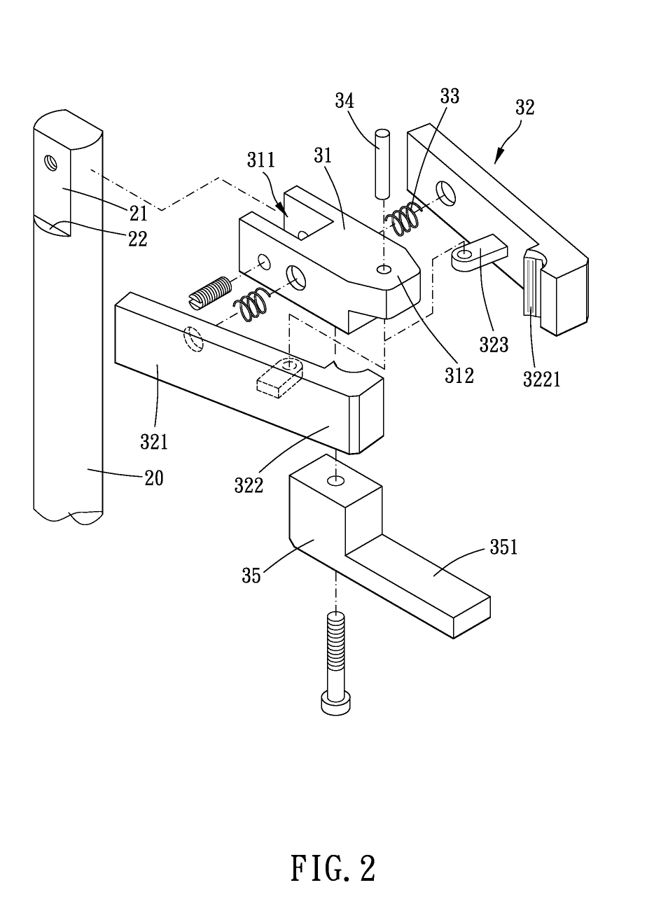

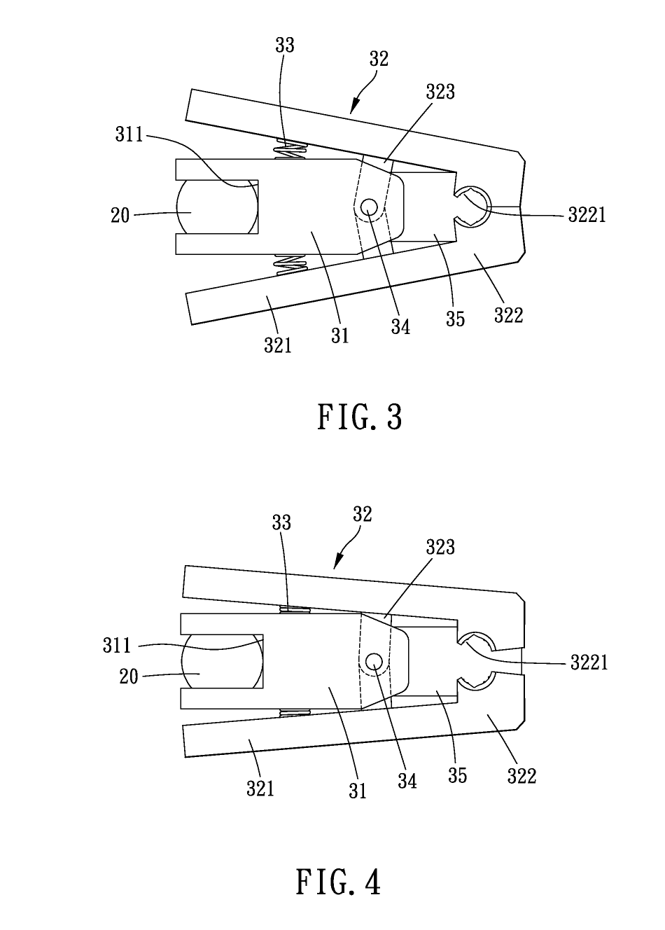

Please refer to FIG. 1 to FIG. 7 for a major embodiment of the present invention. The syringe cap removing device of the present invention is adapted for clamping a cap 43 of a syringe 40 to further remove a needle 42 of the syringe 43. The removing device includes a seat 10, a main body 20, and a clamp 30.

In the present embodiment, the seat 10 includes a base 11, a stand 12, and a support element 13. The base 11 has positioning mechanisms to be positioned on a table, to a periphery of table, on a rack or others. The stand 12 is disposed on the base 11. The support element is detachably disposed on an end of the stand 12 opposite to the base 11. Specifically, the base 11 has a first through hole 111. The stand 12 is rod-shaped to insert through the first through hole 111 and is positioned at the first through hole 11 by a first fastening element 112.

The main body 20 is detachably disposed on the seat 10 with an end thereof. In the present embodiment, the main body 20 is detachably disposed on the support element 13. More specifically, the support element 13 has a second through hole 131. The main body 20 is rod-shaped to insert through the second through hole 131 and is positioned to the second through hole 131 by a second fastening element 132. The clamp 30 is disposed on an end of the main body 20 opposite to the seat 10.

The clamp 30 defines a clamping space to receive the cap 43 of the syringe 40 and to clamp it. In the present embodiment, the clamp includes a base portion 31 and two clamping arms 32. The base portion 31 is disposed on the main body 20. Each clamping arm 32 has a pressing portion 321 and a clamping portion 322 at two opposite ends thereof. The clamping space is enclosed by the two clamping portion 322. Each clamping arm 32 is pivotally disposed on the base portion 31. The connection between the clamping arm 32 and the base portion 31 is located between the pressing portion 321 and the clamping portion 322. The two clamping arms 32 are located at two sides of the base portion 31, and each clamping arm 32 is pushed by an elastic element 33. The elastic element 33 is located between the pressing portion 321 and the connection between the clamping arm 32 and the base portion 31 so that the two clamping portions 322 tend to approach each other. Specifically, each clamping portion 322 includes a cambered face 3221. The two cambered faces 3221 face each other. The clamping space is defined between the two cambered faces 3221. An axial direction is defined by the clamping space. Each cambered face 3221 is formed with a plurality of elongated ribs or grooves arranged spacedly. Each elongated rib or groove has a longitudinal direction parallel to the axial direction. Due to the elongated ribs or the grooves, friction is increased for positioning the cap 43 of the syringe 40. Preferably, in a normal state, the clamping space is substantially equal to or slightly smaller than the size of the cap 43. Thereby, the cap 43 can be positioned to the clamping space without pressing the pressing portion 321.

More practically, the main body 20 is formed with two contacting faces 21 opposite to each other at an end thereof opposite to the seat 10. The base portion 31 has a through 311 for sleeved onto the main body 20 to cover the contacting faces 21. The base portion 31 is screwed with the main body 20. The two contacting faces 21 are recessed relative to other portion of the main body 20 so that a stepped face 22 is formed at an end of each contacting face 21 neat the seat 10 wherein the stepped face 22 is perpendicular to the contacting face 21. The two stepped faces 22 are adapted for being abutted against by the base portion 31 to prevent the base portion 31 from pivoting with respect to the main body 20. The base portion 31 is substantially U-shaped and has a pivot portion 312 for connecting to the two clamping arms 32 at an end opposite to the trough 311. Each clamping arm 32 has an extending arm 323 which extends from a side of the clamping arm 32 facing the base portion 31. The two extending arms 323 overlap the pivot portion 312 of the base portion 31. A pivot pin 34 is inserted through the pivot portion 312 and the two extending arms 32. A first direction is defined as a direction from the opening of the trough 311 to the pivot portion 312. The axial direction of the clamping space is substantially perpendicular to the first direction. Besides, a blocking element 35 is arranged below the base portion 31 and the two clamping arms 32. The blocking element 35 has a blocking portion 351. The blocking portion 351 is right under the clamping space to be abutted against by a tip of the cap 43 to prevent it from falling down.

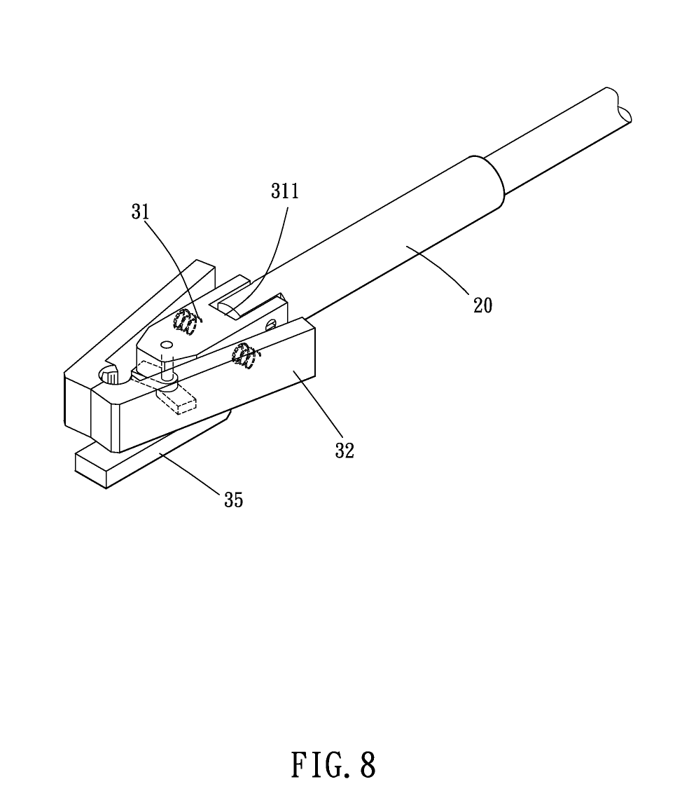

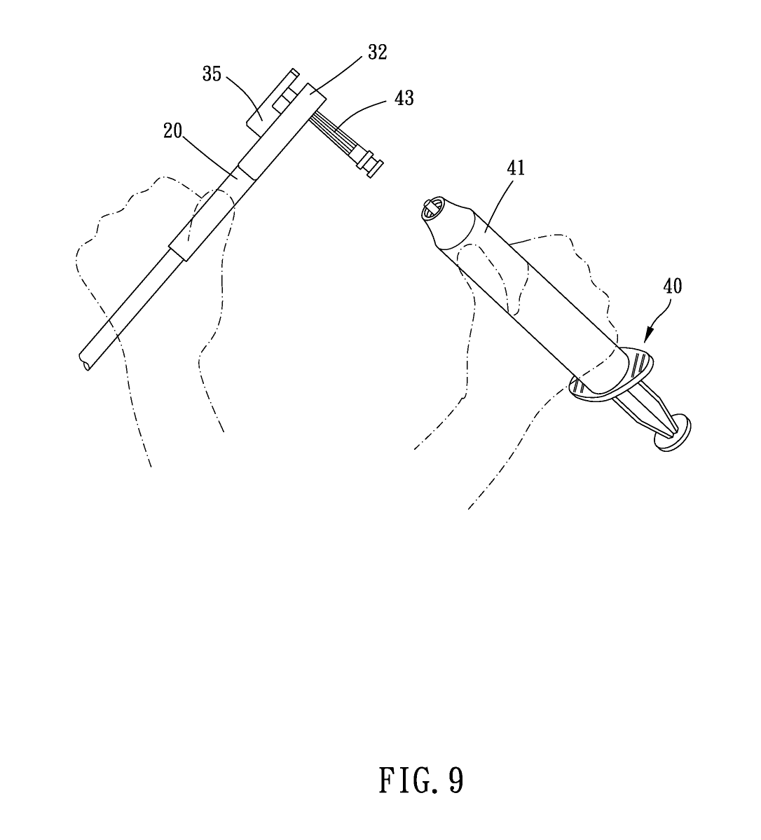

In the present embodiment, the bottom of the trough 311 abuts against the lateral face of the main body 20 to make the main body 20 insert through the trough so that the axial direction of the clamping space is parallel to the longitudinal direction of the main body 20. As a result, the clamp 30 and the main body 20 are L-shaped. However, as shown in FIGS. 8 and 9, the clamp is able to be removed from the main body 20 and installed onto the main body again, so the bottom of the trough 311 can abut against the terminal end of the main body 20 so that the axial direction of the clamping space is perpendicular to the longitudinal direction of the main body 20. That is, the clamp and the main body 20 are parallel to each other or are aligned.

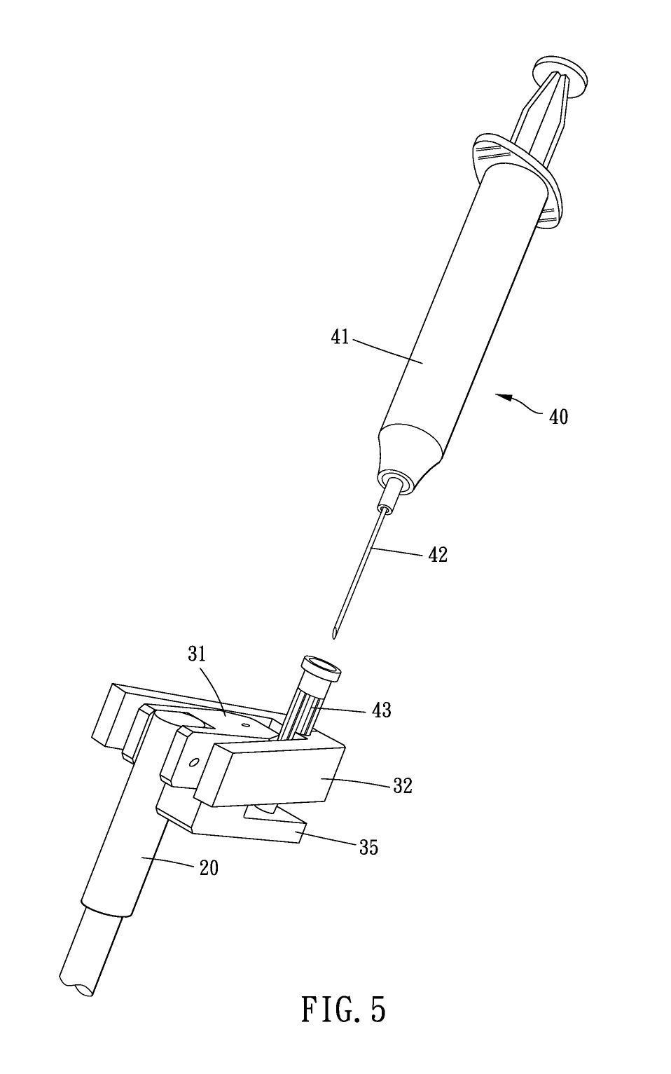

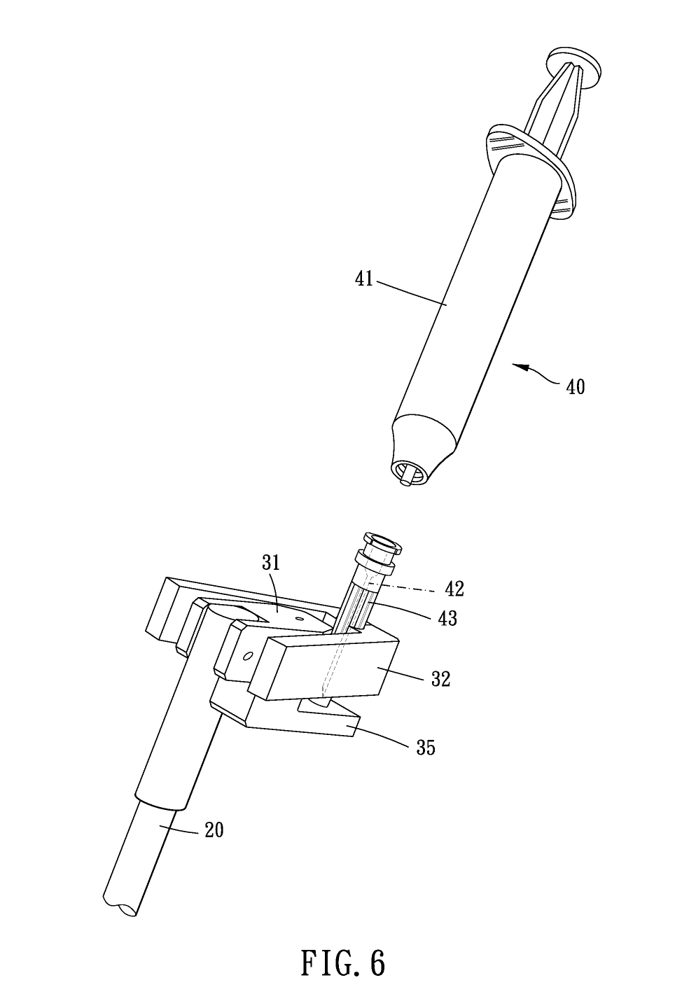

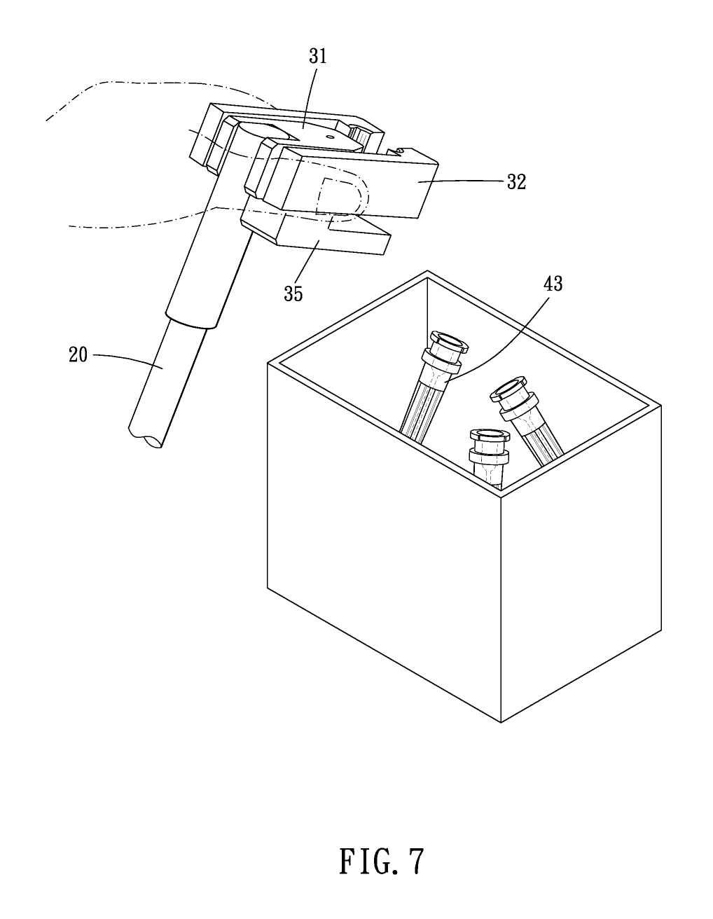

In use, the seat 10 can be positioned on a table, a rack, or other places, and the main body 20 is positioned on the seat 10, as shown in FIG. 1. A user can insert the cap 43 of the syringe 40 into the clamping space so that the cap 43 is temporally positioned in the clamping space, as shown in FIG. 5. Thereafter, the user can hold a syringe 40 to insert the needle 42 thereof into the cap 43 and make the needle 42 positioned to the cap 43. Thus, the cylinder 41 of the syringe 40 can be separated from the needle 42 by rotating the cylinder 41, and then the needle 42 is positioned in the cap 43, as shown in FIG. 6. Thereby, the user can take the clamp to a collecting container and press the pressing portion to open the clamping portions so that the cap 43 with the needle 42 falls into the collecting container for collection, as shown in FIG. 7. Thus, the needle may not be touched by the user.

Also, the present invention can be held by hand. Please refer to FIGS. 8 and 9, the main body 20 can be held by one hand directly, and the syringe 40 is held by the other hand. As a result, the user is able to operate at any place and any direction.

Besides, as shown in FIG. 1, the main body 20 can be rotated to a desired orientation before being positioned by the second fastening element 132.

What is claimed is:

1. A syringe cap removing device, including:
a seat;
a main body, an end thereof being detachably disposed on the seat;
a clamp, an end thereof being disposed on an end of the main body opposite to the seat, the clamp defining a clamping space for clamping a syringe cap.

2. The syringe cap removing device of claim 1, wherein the seat includes a base, a stand, and a support element, the stand is disposed on the base, the support element is detachably disposed on an end of the stand opposite to the base, the main body is detachably disposed on the support element.

3. The syringe cap removing device of claim 2, wherein the base has a first through hole, the stand is rod-shaped to insert through the first through hole and is fastened to the base by a first fastening element, the support element has a second through hole, the main body is rod-shaped to insert through the second through hole and is fastened to the support element by a second fastening element.

4. The syringe cap removing device of claim 1, wherein the clamp includes a base portion and two clamping arms, the base portion is disposed on the main body, each clamping arm has a pressing portion and a clamping portion at two opposite ends, the clamping space is enclosed by the two clamping arms, each clamping arm is pivotally disposed on the base portion at a portion between the pressing portion and the clamping portion, the two clamping arms are located at two sides of the base portion respectively and are pushed by elastic elements, each elastic element is located between the pressing portion and a portion of the clamping arm connecting to the base portion so that the two clamping portions tend to approach each other.

5. The syringe cap removing device of claim 4, wherein each clamping portion includes a cambered face, the two cambered faces face each other.

6. The syringe cap removing device of claim 5, wherein an axial direction is defined by the clamping space, each cambered face is formed with a plurality of elongated ribs or grooves arranged spacedly, a longitudinal direction of each elongated rib or groove is parallel to the axial direction.

7. The syringe cap removing device of claim 4, wherein each clamping arm has an extending arm at an end thereof opposite to the seat, the two extending arms and the base portion overlap and are inserted through by a pivot pin so that the two extending arms are pivotally disposed on the base portion.

8. The syringe cap removing device of claim 4, wherein main body is formed with two contacting faces opposite to each other at an end opposite to the seat, the base portion has a trough sleeved onto the main body to cover the two contacting faces, the base portion is screwed with the main body.

9. The syringe cap removing device of claim 8, wherein the two contacting faces are recessed relative to other portions of the main body so that a stepped face is formed at an end of each contacting face near the seat, the stepped face is substantially perpendicular to the contacting face, the two stepped faces are adapted for being abutted by the base portion so as to prevent the base portion from pivoting with respect to the main body.

10. The syringe cap removing device of claim 8, wherein the base portion is substantially U-shaped, the base portion has a pivot portion at an end thereof opposite to the trough, the pivot portion is pivotally connected with the two clamping arms, a first direction is defined by trough to the pivot portion, an axial direction of the clamping space is substantially perpendicular to the first direction.

11. The syringe cap removing device of claim 10, wherein a bottom of the trough abuts against a terminal end of the main body so that the axial direction of the clamping space is perpendicular to a longitudinal direction of the main body.

12. The syringe cap removing device of claim 10, wherein a bottom of the trough abuts against a lateral face of the main body so that the main body is inserted through the trough, the axial direction of the clamping space is parallel to a longitudinal direction of the main body.

13. The syringe cap removing device of claim 4, wherein a blocking element is arranged below the base portion and the two clamping arms, the blocking element has a blocking portion right below the clamping space for being abutted against by a tip of the syringe cap.

14. A syringe cap removing device, including:
a main body, adapted for being held;
a clamp, the clamp being disposed on an end of the main body, the clamp defining a clamping space for clamping a syringe cap.

15. The syringe cap removing device of claim 14, wherein the clamp includes a base portion and two clamping arms, the base portion is disposed on the main body, each clamping arm has a pressing portion and a clamping portion at two opposite ends, the clamping space is enclosed by the two clamping arms, each clamping arm is pivotally disposed on the base portion at a portion between the pressing portion and the clamping portion, the two clamping arms are located at two sides of the base portion respectively and are pushed by elastic elements, each elastic element is located between the pressing portion and a portion of the clamping arm connecting to the base portion so that the two clamping portions tend to approach each other.

16. The syringe cap removing device of claim 15, wherein each clamping portion includes a cambered face, the two cambered faces face each other.

17. The syringe cap removing device of claim 16, wherein an axial direction is defined by the clamping space, each cambered face is formed with a plurality of elongated ribs or grooves arranged spacedly, a longitudinal direction of each elongated rib or groove is parallel to the axial direction.

18. The syringe cap removing device of claim 15, wherein main body is formed with two contacting faces opposite to each other at an end connecting with the clamp, the base portion has a trough sleeved onto the main body to cover the two contacting faces, the base portion is screwed with the main body.

19. The syringe cap removing device of claim 18, wherein the two contacting faces are recessed relative to other portions of the main body so that a stepped face is formed at an end of each contacting face, the stepped face is substantially perpendicular to the contacting face, the two stepped faces are adapted for being abutted by the base portion so as to prevent the base portion from pivoting with respect to the main body.

20. The syringe cap removing device of claim 15, wherein a blocking element is arranged below the base portion and the two clamping arms, the blocking element has a blocking portion right below the clamping space for being abutted against by a tip of the syringe cap.
